(19) United States
(12) Patent Application Publication
(10) Pub. No.: US 2025/0140366 A1
(43) Pub. Date: May 1, 2025

(54) INFORMATION PROCESSING DEVICE AND PROGRAM STORAGE MEDIUM

(71) Applicant: SEIKO EPSON CORPORATION, Tokyo (JP)

(72) Inventors: Yukiyasu SEKI, SHIOJIRI-SHI (JP); Fumikazu SANO, MATSUMOTO-SHI (JP)

(73) Assignee: SEIKO EPSON CORPORATION, Tokyo (JP)

(21) Appl. No.: 18/928,191

(22) Filed: Oct. 28, 2024

(30) Foreign Application Priority Data
Oct. 30, 2023 (JP) 2023-185489

(51) Int. Cl. G16H 20/30

(52) U.S. Cl. CPC G16H 20/30

(57) ABSTRACT

An information processing device includes a goal setting unit configured to set a first goal for an exercise of a subject, an advice generation unit configured to generate a first advice for the first goal, an output unit configured to output the first goal and the first advice, a motivation estimation unit configured to estimate a motivation of the subject, and an evaluation unit configured to evaluate a motivation of the subject for the first goal. The advice generation unit generates a second advice different from the first advice based on a result of the evaluation by the evaluation unit, and the output unit outputs the second advice.

The present application is based on, and claims priority from JP Application Serial Number 2023-185489, filed Oct. 30, 2023, the disclosure of which is hereby incorporated by reference herein in its entirety.

### BACKGROUND

### 1. Technical Field

The present disclosure relates to an information processing device and a program storage medium.

### 2. Related Art

A device for providing an advice related to sports to the user is known.

In the information processing device disclosed in JP-A-2014-228725, the proficiency of the user for the operation is calculated on the basis of history information related to the operation performed by the user to achieve a predetermined object and property information related to the physical characteristic of the user, and an advice for achieving the object is generated on the basis of the proficiency (see JP-A-2014-228725).

However, in the known technique, the advice is generated on the basis of proficiency, and therefore if the proficiency remains the same, the advice of the same content may be continuously generated, and the advice may be insufficient for the user, for example.

### SUMMARY

To solve the above-mentioned problems, an information processing device according to an aspect includes a goal setting unit configured to set a first goal for an exercise of a subject, an advice generation unit configured to generate a first advice for the first goal, an output unit configured to output the first goal and the first advice, a motivation estimation unit configured to estimate a motivation of the subject, and an evaluation unit configured to evaluate the motivation of the subject for the first goal. The advice generation unit generates a second advice different from the first advice based on a result of the evaluation by the evaluation unit, and the output unit outputs the second advice.

To solve the above-mentioned problems, a program storage medium according to an aspect is configured to store a program, the program causing a computer to execute a first goal setting function of setting a first goal for an exercise of a subject, a first advice generation function of generating a first advice for the first goal, a first output function of outputting the first goal and the first advice, a first estimation function of estimating a motivation of the subject, a first evaluation function of evaluating the motivation of the subject for the first goal, a second advice generation function of generating a second advice different from the first advice based on a result of the evaluation by the first evaluation function, and a second output function of outputting the second advice.

### BRIEF DESCRIPTION OF THE DRAWINGS

FIG. 1 is a diagram illustrating a schematic configuration example including an information processing system an information processing device according to an embodiment.

FIG. 2 is a diagram illustrating an example of a procedure of a process of estimating the motivation on the basis of the frequency of checking the advice according to the embodiment.

FIG. 3 is a diagram illustrating an example of a table used for estimating the motivation on the basis of the exercise time and exercise intensity according to the embodiment.

FIG. 4 is a diagram illustrating an example of a procedure of a process performed in the information processing device according to the embodiment.

### DESCRIPTION OF EMBODIMENTS

An embodiment is described below with reference to the accompanying drawings.

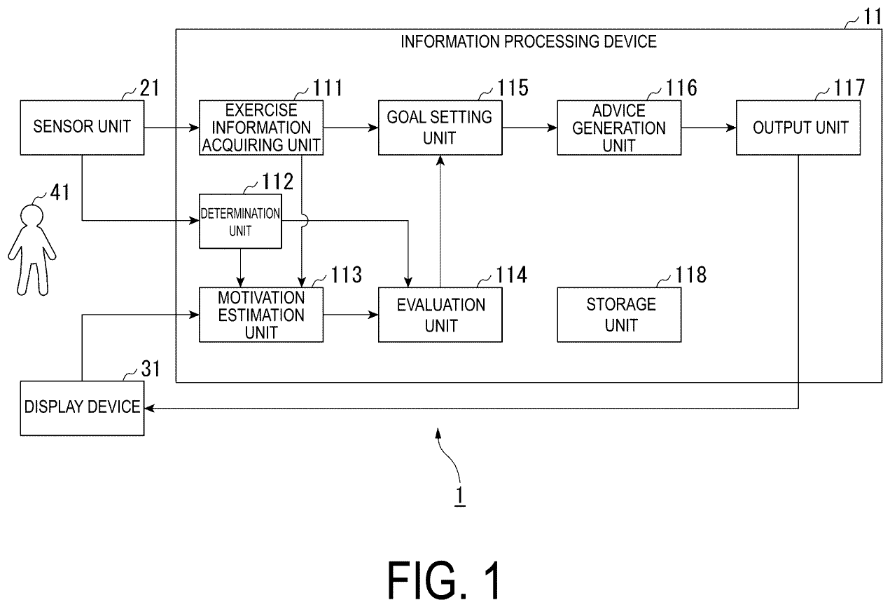

FIG. 1 is a schematic configuration example of an information processing system 1 including an information processing device 11 according to the embodiment.

The information processing system 1 includes the information processing device 11, a sensor unit 21, and a display device 31.

In addition, FIG. 1 illustrates a subject 41 using the information processing system 1. The subject 41 is a person.

Note that the subject 41 may be referred to as a user, for example.

This embodiment describes a case where the information processing device 11, the sensor unit 21, and the display device 31 are separate devices, but as another example, one or both of the sensor unit 21 and the display device 31 may be included in the information processing device 11.

In addition, in this embodiment, one subject 41 is represented for convenience of description, but a plurality of users including the subject 41 may use a common information processing device 11. In the case where there is a plurality of users, the users may be identified with given respective identification information, for example.

The information processing device 11 may be composed of a cloud server device or the like, for example.

The sensor unit 21 includes one or more sensors.

Each sensor detects information related to the subject 41. In this embodiment, this information is information related to the movement of the subject 41.

The movement of the subject 41 may be the movement of body parts visible to the outside world, such as the head, torso, arms, hands, legs and feet of the subject 41, or the movement of internal body parts of the subject 41, such as the heart and respiratory system of the subject 41, for example.

The sensor unit 21 transmits information detected by one or more sensors to the information processing device 11.

The information may be information of the detection result of each sensor, or combined information of detection results of two or more different sensors.

Note that the communication between the sensor unit 21 and the information processing device 11 may be radio communication or wired communication, for example.

The sensors may be various sensors, such as an inertial measurement unit (IMU), an optical heart rate sensor using photoplethysmography (PPG), and a global positioning system (GPS), for example.

Each sensor is attached to the subject 41 when used.

The sensor may be attached to the subject 41 by being fixedly attached to the body of the subject 41, or being held in the hand of the subject 41, or, being housed in the clothing or bag of the subject 41 or the like, for example.

As a specific example, it is possible to use a sensor incorporated in a predetermined device such as a smartphone, personal computer, tablet, and watch, or a dedicated sensor fixedly attached to a predetermined portion such as the head, body, arm, hand, leg, and foot of the subject 41. The dedicated sensor may be attachable to and detachable from the body of the subject 41, for example.

Note that in some cases a sensor such as an IMU, PPG, GPS, and an acceleration sensor may be incorporated in the device such as a smartphone, for example. In addition, in some cases a pedometer, heart rate monitor, pulse rate meter or the like using a predetermined sensor may be incorporated in the device such as a smartphone, for example.

The display device 31 includes a screen that displays predetermined information. In this embodiment, this information is input from the information processing device 11 to the display device 31.

In addition, in this embodiment, the display device 31 may transmit to the information processing device 11 information related to the display of the predetermined information, a matter received from the subject 41 and the like. The display device 31 may include a touch panel screen with a function of displaying information and a function of receiving the operation of the subject 41, for example.

Note that the communication between the display device 31 and the information processing device 11 may be radio communication or wired communication, for example.

The display of information by the display device 31 may be controlled by a predetermined application, for example.

As a specific example, the display device 31 may be a device such as a smartphone, personal computer, tablet, and watch.

Note that the same device may have both the sensor function of the sensor unit 21 and the function of the display device 31. In this case, the sensor unit 21 and the display device 31 may be configured as an integrated device.

In addition, the same device may have all of the sensor function of the sensor unit 21, the function of the display device 31, and the function of the information processing device 11. In this case, the sensor unit 21, the display device 31 and the information processing device 11 may be configured as an integrated device.

The information processing device 11 includes an exercise information acquiring unit 111, a determination unit 112, a motivation estimation unit 113, an evaluation unit 114, a goal setting unit 115, an advice generation unit 116, an output unit 117, and a storage unit 118.

In this embodiment, the information processing device 11 is configured with a computer. The information processing device 11 performs various processes and controls when a processor such as a CPU (Central Processing Unit) executes a predetermined program.

Note that FIG. 1 illustrates with an arrow an example of a procedure of exchanging information regarding the sensor unit 21, the display device 31, and the components of the information processing device 11, but this example is not limitative, and information may be exchanged between given components depending on the need for the information processing.

The exercise information acquiring unit 111 acquires information that is transmitted from the sensor unit 21 and received by the information processing device 11, and outputs, based on that information, the information related to the exercise performed by the subject 41 to one or both of the motivation estimation unit 113 and the goal setting unit 115.

The exercise information acquiring unit 111 may output the information acquired from the sensor unit 21 to the motivation estimation unit 113 or the goal setting unit 115 as it is, or output information obtained by processing that information to the motivation estimation unit 113 or the goal setting unit 115, for example.

In this embodiment, the information acquired by the exercise information acquiring unit 111 is information related to the movement of the subject 41, and is used as information related to the exercise of the subject 41 in this embodiment.

As an example, the exercise information acquiring unit 111 may acquire the information about a general exercise performed by the subject 41, or as another example, the exercise information acquiring unit 111 may acquire information about a specific exercise performed by the subject 41. That is, the exercise in this embodiment may be a general exercise, or a specific exercise.

The specific exercise may be a specific sport exercise, for example.

The exercise performed by the subject 41 is not limited and may be, for example, sporting exercise such as marathon, golf, tennis, football, and the like. In addition, the exercise may also include, for example, muscle training for personal health. Note that personal muscle training may also be regarded as a type of sport.

The exercise may or may not include walking or running in daily life.

Note that the information processing device 11 may determine and distinguish or not distinguish between the state where the subject 41 is performing a specific exercise and the state where the subject 41 is not performing a specific exercise.

In the case where the information processing device 11 determines the state where the subject 41 is performing a specific exercise and the state where the subject 41 is not performing a specific exercise, a given component may be provided with a function of performing the determination, and for example, the exercise information acquiring unit 111, the determination unit 112, a motivation designate unit 113 or the like may be provided with that function.

As an example, when the sensor of the sensor unit 21 attached to the subject 41 is in an on state, it may be regarded as a state where the subject 41 is performing a specific exercise. Specifically, when the information from the sensor is input to the information processing device 11, it may be regarded as a state where the subject 41 is performing a specific exercise.

Note that in this embodiment, when the sensor of the sensor unit 21 is not attached to the subject 41, the sensor is turned off, and the information from the sensor is not input to the information processing device 11.

As a specific example, in a case where the specific exercise is a marathon competition, the subject 41 attaches the sensor to the body of the subject 41 and turns on the sensor when starting running the marathon, and turns off the sensor when finishing running the marathon, for example.

As another example, when a specific movement of the subject 41 is detected by the sensor of the sensor unit 21, it may be regarded as a state where the subject 41 is performing a specific exercise. The specific movement of the subject 41 may be detected by a specific sensor, for example.

As a specific example, in a case where the specific exercise is a marathon competition, and the information processing device 11 determines that the subject 41 is running based on the detection information of the sensor, it may be regarded as a state where the subject 41 is running the marathon, for example.

Note that the information processing device 11 may determine the presence/absence of the specific movement of the subject 41, and may monitor the specific movement of the subject 41 at all times, such that it determines that the subject 41 is performing a specific exercise when the detection value of the sensor corresponding to the movement is greater than a predetermined threshold value, whereas it determines that the subject 41 is not performing a specific exercise in other cases, for example.

As another example, it is possible to use a configuration the subject 41 directly or indirectly inputs information about the start time of a specific exercise and the finish time of the exercise to the information processing device 11. This input may be performed by directly operating the information processing device 11 by the subject 41, or by operating a device such as a smartphone by the subject 41 to transmit information from the device to the information processing device 11, for example.

Note that the start time of a specific exercise and the finish time of the exercise may not necessarily be simultaneously input to the information processing device 11, but may be input to the information processing device 11 at respective timings that differ in time.

As a specific example, in a case where the specific exercise is a marathon competition, the information of the timing of starting the marathon and the information of the timing of finishing the marathon may be input to the information processing device 11, for example.

The determination unit 112 acquires the information that is transmitted from the sensor unit 21 and received by the information processing device 11, and performs determination regarding the state of the subject 41 on the basis of that information.

The determination unit 112 outputs the information of the determination result to the motivation estimation unit 113. In addition, the determination unit 112 may output the information of the determination result to the evaluation unit 114.

This determination may be a determination regarding various states.

Note that the determination unit 112 may be referred to as state determination unit, state measurement unit or the like, for example.

In this embodiment, regarding the exercise of the subject 41, an ideal goal, which is an ideal target of the exercise, is set. The ideal goal may be input and set by the subject 41 or another person by using a predetermined application, for example. The application to be used may be the application of the information processing device 11, or the application of other devices, for example. The other devices may be the sensor unit 21 or the display device 31, for example.

As another example, the ideal goal may be automatically set by the information processing device 11 or other devices. The other devices may be the sensor unit 21 or the display device 31, for example. Various methods of automatically setting the ideal goal may be used, such as a method of determining and setting the ideal goal based on the information related to the body of the subject 41 such as the age of the subject 41, or a computation based on a change in that information, for example.

The ideal goal may be set as necessary for each type of the exercise, and it is possible to set goals such as the time, score, or load to be cleared, and further it is possible to set the period until achieving the goals, for example.

As a specific example, in the case where the exercise of the subject 41 is a marathon competition, an ideal goal value for the time of a marathon may be set as the ideal goal, and further the ideal period until achieving that time may be set to be included in the ideal goal. As an example, the information processing device 11 may set as the ideal goal the general fastest record time, average record time or the like corresponding to the body information for a marathon on the basis of the body information such as the age of the subject 41.

Note that in this embodiment, in the information processing device 11, the function of setting the ideal goal is provided in the goal setting unit 115. When automatically setting the ideal goal, the goal setting unit 115 may refer to one or both of the information from the exercise information acquiring unit 111 and the information of the determination result obtained by the determination unit 112, for example.

As another example, the function of setting the ideal goal may be provided in the determination unit 112 or in another given component illustrated in the example of FIG. 1, or may be provided as an independent component not illustrated in the example of FIG. 1.

In addition, in this embodiment, regarding the exercise of the subject 41, an intermediate goal, which is an intermediate target of the exercise, is set. In this embodiment, the intermediate goal is set by the goal setting unit 115 of the information processing device 11.

The intermediate goal is an intermediate goal before achieving the ideal goal, and is a goal corresponding to the state between the current state of the subject 41 and the ideal goal state.

As a first example, the determination unit 112 may determine the current state relative to the ideal goal state regarding the exercise of the subject 41.

As a second example, the determination unit 112 may determine the item required for achieving the ideal goal state regarding the exercise of the subject 41. The item may be the item on the movement of the subject 41.

As a third example, regarding the exercise of the subject 41, the determination unit 112 may determine whether the intermediate goal state has been achieved, and may determine the period taken for the achievement in the case where the state has been achieved.

In the above-described determinations, it is possible to determine the difference between the state of the goal such as the ideal goal and intermediate goal, and the current state of the subject 41, for example.

As a specific example, an example case where the exercise of the subject 41 is a marathon competition is described below.

It is assumed that the ideal goal is that the subject 41 runs 42.195 km in four hours.

In the determination of the first example, the determination unit 112 may determine as the current state the time that is measured with the detection result of the sensor when the subject 41 is running a marathon, for example. The current time may not be the exactly current time, and a slightly past time such as the time of one previous marathon may be regarded as the current time, for example. The sensor may include a GPS sensor that detects the location information.

Note that the determination unit 112 may not exactly determine the current state, and the current state estimated by a predetermined estimation method may be regarded as a determination result, for example. As a specific example, it is possible to use an estimation method of estimating the time based on the exercise amount of the marathon performed by the subject 41.

In the determination of the second example, the item may be an item that is necessary to achieve the ideal target state, i.e. a form that improves time and approaches the ideal state in a marathon. Details of such form include the ground contact time, which is generally preferably be shorter, or the vertical movement, which is generally preferably be smaller, for example. Other items may be used as such items.

Such items may be determined in advance for each sport, for example.

In the determination of the third example, the period taken for the subject 41 to achieve the one previous intermediate goal state is used as the period taken for achieving the one previous intermediate goal state.

In this embodiment, as the period taken for achieving the one previous intermediate goal state, it is possible to determine the period from the presentation of the latest advice presented before the achievement of the one previous intermediate goal state, to the time point of the achievement of the one previous intermediate goal state, for example.

Note that as another example, in the case where the subject 41 has started the exercise at an initial timing, and there is no intermediate goal closer to the start point than the one previous intermediate goal, i.e., the one previous intermediate goal is the first intermediate goal after the start point, it is possible to determine the period from the start point to the time point of the achievement of the one previous intermediate goal state. In addition, in the case where there is the second to one previous intermediate goal, which is the intermediate goal one before the one previous intermediate goal, it is possible to determine the period from the time point of the achievement of the second to one previous intermediate goal state to the time point of the achievement of the one previous intermediate goal state.

The motivation estimation unit 113 estimates the motivation of the subject 41.

In this embodiment, the motivation estimation unit 113 estimates the motivation of the subject 41 on the basis of one or more of the information from the exercise information acquiring unit 111, the information from the determination unit 112, and the information from the display device 31.

The motivation estimation unit 113 outputs the information about the estimation result of the motivation to the evaluation unit 114.

In this embodiment, the motivation estimation unit 113 estimates the motivation of the subject 41 on the basis of one or more of a plurality of parameters regarding the exercise of the subject 41. The plurality of parameters may include one or more of the exercise time, the number of times of the exercise, exercise frequency, exercise intensity, the period taken to achieve the one previous intermediate goal, and the frequency of checking the advice, for example. As an example, the plurality of parameters may be four parameters of the exercise time, the number of times of the exercise or the exercise frequency, the exercise intensity, and the frequency of checking the advice, but this is not limitative.

Note that both the number of times of the exercise and the exercise frequency may be used, or one of the number of times of the exercise and the exercise frequency may be used.

The motivation estimation unit 113 has a function of generating a predetermined exercise-related information. In this embodiment, the exercise-related information is the exercise time, the number of times of the exercise or the exercise frequency, the exercise intensity, and the period taken to achieve the one previous intermediate goal state.

As the exercise time, it is possible to use the daily exercise time, or the exercise time of another period, for example.

The motivation estimation unit 113 calculates the time over which there is a movement with an intensity of a certain level or greater in the measurement time of the sensor of the sensor unit 21, and sets this time as the exercise time, for example. The time over which there is a movement with an intensity of a certain level or greater may be the time over which the acceleration or the angular velocity detected by a sensor such as an IMU is a certain value or greater, for example.

The number of times of the exercise may be the number of times of the exercise in a certain period, for example. The certain period may be any period.

The motivation estimation unit 113 calculates the number of times of the exercise on the basis of the information about the measurement date of the sensor of the sensor unit 21, for example.

As a specific example, the motivation estimation unit 113 may estimate that the number of times of the exercise is large when the number of times of the exercise in one week is seven or greater.

The exercise frequency may be the number of times of the exercise in a certain period, for example. The certain period may be any period.

The motivation estimation unit 113 calculates the exercise frequency on the basis of the information about the measurement date of the sensor of the sensor unit 21, for example.

As a specific example, the motivation estimation unit 113 may estimate that the exercise frequency is high when there are the measurement dates for seven days in one week as the measurement date on which the subject 41 has performed the exercise.

As the exercise intensity, it is possible to use the daily exercise load, or the exercise load of another period, for example.

The motivation estimation unit 113 may calculate the exercise load on the basis of one or more of the acceleration or angular velocity detected by an IMU sensor, the heart rate detected by a heart rate monitor, or the pulse detected by a pulse rate meter, for example.

As a specific example, the motivation estimation unit 113 may calculate the average pulse of the subject 41 during the exercise time, and estimate that the exercise load is high, i.e., the exercise intensity is high when the average pulse is higher than a predetermined threshold value.

The period taken to achieve the one previous intermediate goal state may be the result of calculation of the number of days from the date when the advice is presented to the subject 41, to the achievement of the one previous intermediate goal state. This calculation may be performed on the basis of the advice history of the application that presents the advice, for example.

The motivation estimation unit 113 calculates the number of days taken to achieve the one previous intermediate goal state on the basis of the determination result of the determination whether the one previous intermediate goal state has been satisfied based on the measurement data of the predetermined sensor, for example. The determination result may be the determination result obtained by the determination unit 112.

As a specific example, the motivation estimation unit 113 may estimate that the period is short when the number of days taken to achieve the one previous intermediate goal state is three days, and may estimate that the period is long when the number of days is one month.

The motivation estimation unit 113 has a function of generating predetermined behavior information related to the subject 41. In this embodiment, the behavior information is the frequency of checking the advice.

The frequency of checking the advice may be the frequency based on the number of times the screen displaying the advice is opened. The number of times may be calculated on the basis of the operation history of the application that displays the advice. Note that the display of advice may be performed together with the display of the goal.

As a specific example, the motivation estimation unit 113 may calculate the number of times the corresponding screen is checked in one day as the frequency of checking the advice.

When generating the frequency of checking the advice, the motivation estimation unit 113 may refer to information such as the timing, the number of times and the frequency of the advice generated by the advice generation unit 116, for example.

Note that the number of times of checking the advice may be used together with or instead of the frequency of checking the advice, for example.

In this embodiment, the information related to the exercise of the subject 41 is detected by the sensor of the sensor unit 21 as the exercise information, and the information related to information other than the exercise of the subject 41 is detected by another application as the behavior information.

The behavior information may be generated in response to a predetermined operation performed by the subject 41 in a predetermined application, for example. The predetermined operation may be an operation for viewing the advice.

Note that in this embodiment, the function of the sensor used for detecting the exercise information and the function of the application used for detecting the behavior information are separate functions, but those functions may be provided in the same device such as a smartphone.

In this embodiment, for convenience of description, the motivation estimation unit 113 generates the predetermined exercise-related information on the basis of the exercise information acquired by the exercise information acquiring unit 111 and as necessary the information from the determination unit 112, and generates the behavior information on the basis of the information such as the operation history from the display device 31.

In this manner, in this embodiment, the exercise information and the exercise-related information are distinguished from each other for convenience of description, and the exercise-related information is generated from the exercise information, but the exercise-related information may be considered to be included in the exercise information because the exercise-related information is also the information related to the exercise of the subject 41, for example.

Note that the exercise information and the exercise-related information may be referred to as other names, for example.

In this embodiment, the motivation estimation unit 113 estimates the motivation by dividing it into a plurality of segments.

The number of motivation levels may be, but not limited to, three levels such as high, middle and low, or a plurality of levels such as ten levels, for example. In addition, the number of motivation levels may be two levels.

As a general tendency, it is estimated that the longer the exercise time, the higher the motivation, and that the shorter the exercise time, the lower the motivation.

As a general tendency, it is estimated that the larger the number of times of the exercise or the exercise frequency, the higher the motivation, and that the smaller the number of times of the exercise or the exercise frequency, the lower the motivation.

As a general tendency, regarding the exercise intensity, it is estimated that the higher the load, the higher the motivation, and that the lower the load, the lower the motivation.

As a general tendency, it is estimated that the higher the frequency of checking the advice, the higher the motivation, and that the lower the frequency of checking the advice, the lower the motivation.

As a general tendency, it is estimated that the shorter the period taken to achieve the one previous intermediate goal, the higher the motivation, and that the longer the period taken to achieve the one previous intermediate goal, the lower the motivation. It should be noted that depending on the level of the intermediate goal, the goal may be difficult for the subject 41 to achieve, and as such estimation results different from the above-described estimation may be more preferable.

As an aspect, the motivation estimation unit 113 may determine the estimation result of the motivation on the basis of the value of one of a plurality of parameters. In this case, for example, the estimation result of the motivation may be determined on the basis of the relationship between the value of that parameter and one or more predetermined threshold values.

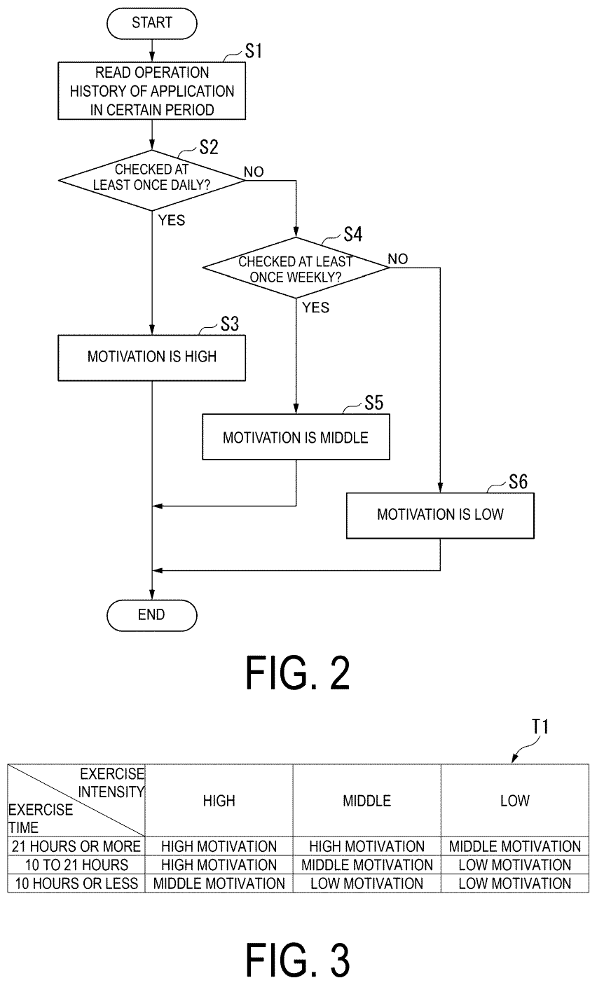

FIG. 2 illustrates an example of a procedure of a process of estimating the motivation on the basis of the frequency of checking the advice according to the embodiment.

In the example illustrated in FIG. 2, the motivation estimation unit 113 performs the processes of step S1 to step S6.

At step S1, for an application for checking the advice that is operated by the subject 41, the motivation estimation unit 113 reads the operation history in a certain period of the application, and proceeds to step S2.

At step S2, the motivation estimation unit 113 determines whether the subject 41 checks the advice one or more times every day on the basis of the read operation history. When the motivation estimation unit 113 determines that the subject 41 checks the advice one or more times every day, i.e., YES in FIG. 2, it proceeds to step S3, otherwise, i.e., NO in FIG. 2, it proceeds to step S4.

In this case, the fact that the advice is checked one or more times every day is used as the threshold value.

In this embodiment, when the advice is viewed by the subject 41, it is considered that the subject 41 has checked the advice.

At step S3, the motivation estimation unit 113 estimates that the motivation is high, and the processing of this procedure is terminated.

At step S4, the motivation estimation unit 113 determines whether the subject 41 checks the advice one or more times weekly on the basis of the read operation history. When the motivation estimation unit 113 determines that the subject 41 checks the advice one or more times weekly, i.e., YES in FIG. 2, it proceeds to step S5, otherwise, i.e., NO in FIG. 2, it proceeds to step S6.

In this case, the fact that the advice is checked one or more times weekly is used as the threshold value.

At step S5, the motivation estimation unit 113 estimates that the motivation is a middle level, and the processing of this procedure is terminated. Note that the middle level may be referred to as common, for example.

At step S6, the motivation estimation unit 113 estimates that the motivation is low, and the processing of this procedure is terminated.

The processing procedure illustrated in FIG. 2 is an example, and other processing procedure of estimating the motivation may be used.

For example, the motivation estimation unit 113 may determine whether the subject 41 checks the advice one or more times monthly, and estimate that the motivation is low when the subject 41 does not check the advice one or more times monthly.

Alternatively, the motivation estimation unit 113 may determine the estimation result of the motivation on the basis of the values of two or more parameters among a plurality of parameters. In this case, for example, for a combination of values of two or more parameters, the estimation result of the motivation may be determined on the basis of the relationship with a predetermined threshold value.

FIG. 3 illustrates an example of a table T1 used for estimating the motivation on the basis of the exercise time and the exercise intensity according to the embodiment.

Note that the information of the table T1 may be stored in the storage unit 118, for example.

The example of the table T1 has three columns of the exercise time, namely, 21 hours or more weekly, 10 to 21 hours weekly, and 10 hours or less weekly. In this case, 10 to 21 hours is a time longer than 10 hours and shorter than 21 hours.

In addition, the example of the table T1 has three columns of the exercise intensity, namely high, middle and low.

In this example, when both the exercise time and the exercise intensity are determined, one column is determined, and the estimation result of the motivation is determined.

In the example of the table T1, each column indicates the high motivation indicating that the motivation is high, the middle motivation indicating that the motivation is middle, or the low motivation indicating that the motivation is low.

In this manner, the motivation estimation unit 113 may estimate the motivation corresponding to the combination of the exercise time and the exercise intensity on the basis of the table T1.

Note that in the example illustrated in FIG. 3, the motivation is estimated on the basis of two parameters, but as another example, the motivation may be estimated on the basis of three or more parameters.

For example, in the case where the motivation is estimated by one parameter such as the exercise time, even when the subject 41 has performed only easy exercise with low intensity for a long time, it is estimated that the motivation is high because of the long exercise time. In view of this, by estimating the motivation by two parameters such as the exercise time and the exercise intensity as in this example, the accuracy of the estimation can be improved.

In the example illustrated in FIG. 3, for example, the motivation is estimated to be high when the exercise with high exercise intensity has been performed for 21 hours or more in one week, whereas the motivation is estimated to be middle when the exercise with the middle exercise intensity has been performed for 10 to 21 hours in one week.

Alternatively, the motivation estimation unit 113 may determine the estimation result of the motivation by calculating an overall score by weighting the values of two or more of a plurality of parameters.

In this case, the weighting may include equal weighting.

In addition, the range of scores may be, but not limited to, a linear range of 0 to 100 points, or other ranges of scores, for example.

As an example, the motivation estimation unit 113 handles all parameters as equal scores.

As a specific example, the motivation estimation unit 113 may handle each of the four parameters as scores of a total of 25 points, and calculate the scores out of 100 by adding up the scores. The four parameters may be the exercise time, any of the number of times of the exercise and the exercise frequency, the exercise intensity, or the frequency of checking the advice, for example.

As another example, the motivation estimation unit 113 may handle the scores by increasing the weight of the parameter that is considered to be especially directly related to the motivation among the plurality of parameters. Note that the weights of the parameters may be set in advance, for example.

As a specific example, in the case where the four parameters such as the exercise time, any of the number of times of the exercise and the exercise frequency, the exercise intensity, and the frequency of checking the advice are used, the motivation estimation unit 113 may calculate the scores out of 100 such that the exercise time, any of the number of times of the exercise or the exercise frequency, and the exercise intensity that are related to the actual exercise among the four parameters are each provided with increased weights and handled as a total of 30 points, while the frequency of checking the advice is handled as a total of 10 points.

Note that the above-described method of calculating the point of the motivation for each parameter can more easily calculate the comprehensive score of the motivation in comparison with the method of determining the motivation for each parameter in a stepwise manner such as high, middle and low.

In this embodiment, the motivation estimation unit 113 estimates the motivation of the subject 41, but the motivation estimation unit 113 may further estimate the proficiency of the subject 41 for the goal, for example. The proficiency may be estimated on the basis of the exercise history of the subject 41 for the goal. This goal is an ideal goal at the first time, and is an intermediate goal set at the one previous time at the time other than the first time.

For example, the proficiency may be used to adjust the motivation.

Note that the proficiency may not be used.

The evaluation unit 114 performs a predetermined evaluation on the basis of the information from the motivation estimation unit 113 to thereby generate the evaluation result based on the estimation result of the motivation. When performing the evaluation, the evaluation unit 114 may also refer to the information from the determination unit 112 together with the information from the motivation estimation unit 113.

The evaluation unit 114 outputs the information about the evaluation result to the goal setting unit 115.

Various methods may be used as the method of evaluation performed by the evaluation unit 114.

For example, on the basis of the estimation result of the motivation of the subject 41 for a predetermined goal, the evaluation unit 114 may evaluate whether the motivation of the subject 41 is appropriate for the level of the goal, or evaluate its appropriateness. This goal may be an ideal goal, or an intermediate goal.

As a specific example, the following considers a case where a goal of running a full marathon in four hours has been set, and the estimated result of the motivation for the level of the goal has been obtained as high, middle or low. This goal is an ideal goal, for example.

In this case, when the estimation result of the motivation is high, the evaluation unit 114 may evaluate that the motivation is sufficient for the level of the goal. In addition, when the estimation result of the motivation is middle, the evaluation unit 114 may evaluate that the motivation is slightly insufficient for the level of the goal, for example. In addition, when the estimation result of the motivation is low, the evaluation unit 114 may evaluate that the motivation is insufficient for the level of the goal, for example.

As another specific example, the following considers a case where a goal of running a full marathon in five hours has been set, and the estimated result of the motivation for the level of the goal has been obtained as high, middle or low. This goal is an intermediate goal, which is a goal with a level lower than an ideal goal, for example.

In this case, when the estimation result of the motivation is high, the evaluation unit 114 may evaluate that the motivation is sufficient for the level of the goal. In addition, when the estimation result of the motivation is middle, the evaluation unit 114 may evaluate that the motivation is sufficient for the level of the goal. In addition, when the estimation result of the motivation is low, the evaluation unit 114 may evaluate that the motivation is slightly insufficient for the level of the goal.

The goal setting unit 115 sets a predetermined goal on the basis of one or both of the information from the exercise information acquiring unit 111 and the information from the evaluation unit 114.

The goal setting unit 115 outputs the information related to the set goal to the advice generation unit 116.

In this case, the goal to be set may be an ideal goal, or an intermediate goal, for example.

In addition, two more intermediate goals may be set.

As an example, it is possible to set an ideal goal first by the goal setting unit 115, and then an intermediate goal for the ideal goal.

As another example, it is possible to set an ideal goal first by the goal setting unit 115, and then a first intermediate goal for the ideal goal, and then, a second intermediate goal for the first intermediate goal. Further, likewise, three or more intermediate goals may be set by the goal setting unit 115.

In this embodiment, the one previous goal with respect to the second intermediate goal, which is the intermediate goal set second, is the first intermediate goal, which is the intermediate goal set first. In addition, the one previous goal with respect to the third intermediate goal, which is the intermediate goal set third, is the second intermediate goal, which is the intermediate goal set second. In addition, the same applies to the subsequent intermediate goals.

Note that the intermediate goal is a goal corresponding to the state between the current state of the subject 41 and the ideal goal state, and is a middle goal before achieving the ideal goal.

The goal setting unit 115 may determine the current state of the subject 41 on the basis of the information from the exercise information acquiring unit 111.

As another example, the information of determination result of the current state of the subject 41 obtained by the determination unit 112 may be output from the determination unit 112 to the goal setting unit 115, and referred to by the goal setting unit 115.

The goal setting unit 115 sets an ideal goal on the basis of the information from the exercise information acquiring unit 111, for example. Specifically, the goal setting unit 115 sets an ideal goal on the basis of the information related to the exercise of the subject 41.

As another example, the goal setting unit 115 may set an ideal goal on the basis of other information, or set a predetermined ideal goal.

The goal setting unit 115 sets an intermediate goal on the basis of the information from the evaluation unit 114, for example. Specifically, the goal setting unit 115 sets an intermediate goal on the basis of the evaluation result based on the estimation result of the motivation of the subject 41.

In general, the intermediate goal is set on the basis of the degree of the motivation for the one previous goal. As an example, assuming that the greater the exercise amount of the subject 41, the higher the motivation, a more difficult intermediate goal may be set.

Further, the intermediate goal may be set in consideration of the period taken to achieve the one previous goal.

In this embodiment, the period taken to achieve the one previous goal may be determined by the determination unit 112, referred to in the estimation of the motivation estimation unit 113, and reflected in the evaluation result of the evaluation unit 114.

As another example, the information of the determination result of the determination unit 112 may be output from the determination unit 112 to the goal setting unit 115. In this case, the goal setting unit 115 may refer to the information from the determination unit 112 when setting the goal.

In this manner, how the level of the intermediate goal to be set is close to the level of the ideal goal may be determined in consideration of two types of parameters, such as the motivation of the subject 41, and the period taken for achieving the preceding intermediate goal, for example.

In this case, when the subject 41 has achieved the intermediate goal, the next intermediate goal is determined in accordance with the motivation of the subject 41 and the period taken for achieving the one previous intermediate goal.

For example, when the motivation of the subject 41 for the one previous intermediate goal is high, the next intermediate goal may be set to a goal closer to the ideal goal than when the motivation is not high.

It should be noted that when the subject 41 has continuously performed the exercise, but the period taken for achieving the intermediate goal is long or the intermediate goal is not achieved, the goal setting unit 115 may determine that it is difficult to achieve the goal despite the motivation of the subject 41, and adjust the next intermediate goal to a goal closer to the current state of subject 41.

On the other hand, for example, when the motivation of the subject 41 for the one previous intermediate goal is low, the next intermediate goal may be set to a goal closer to the current state of the subject 41 than when the motivation is not low.

It should be noted that when the period taken for achieving the intermediate goal is short, the goal setting unit 115 may determine that it is easy to achieve the goal although the motivation of the subject 41 is low, and adjust the next intermediate goal to a goal closer to the ideal goal.

In this manner, the goal setting unit 115 determines the ease of achieving the goal on the basis of the motivation of the subject 41 and the period taken for achieving the preceding intermediate goal, and sets the next goal on the basis of this determination result such that the subject 41 can achieve the goal while maintaining the motivation of the subject 41, for example.

As a specific example, the following considers a case where the goal is to reduce the marathon time.

In this case, for example, the next intermediate goal may be set by using the percentage [%] of approaching the ideal goal for the current state of the subject 41.

As an example, the current state of the subject 41 is a time of six hours, which is considered to be 0 [%]. In addition, the ideal goal state is the time of four hours, which is considered to be 100 [%]. At this time, the next intermediate goal is the time of five hours 55 minutes, the percentage [%] of approaching the ideal goal is approximately 4.2 [%].

In this embodiment, the next intermediate goal is not set when an ideal goal is set in an initial state, but as another example, when setting an ideal goal in an initial state, the goal setting unit 115 may set the next intermediate goal based on the ideal goal simultaneously with or immediately after it.

In this case, the next intermediate goal may be a goal corresponding to a result obtained by equally dividing by a predetermined period the state change until the achievement of the ideal goal.

For example, only for the initial use, the goal setting unit 115 may set an equally divided intermediate goal on the basis of the ideal goal and its period in accordance with the result obtained by dividing by the period the state change until the achievement of the ideal goal.

As a specific example, the current state of the subject 41 is the time of six hours, which is considered to be 0 [%]. In addition, the ideal goal state is the time of four hours, which is considered to be 100 [%]. In addition, it is assumed that the period until achieving the ideal goal from the current state is set to two years.

In this case, the intermediate goal is set such that the ideal goal is achieved after two years. Specifically, in the case of the equal division, from a computation of {120 minutes/24 months}, when the time is reduced by 5 minutes for each month, the ideal goal will be achieved after two years. In this manner, as the next goal, the goal setting unit 115 sets the intermediate goal of achieving the time of five hours 55 minutes after one month.

In reality, in some cases the subject 41 may not achieve the next intermediate goal.

In this case, the goal setting unit 115 may perform one or both of a process of changing the next intermediate goal, and a process of changing the due date by which the next intermediate goal is to be achieved.

For example, if the due date for the next intermediate goal passes while the subject 41 has not yet achieved the next intermediate goal, the goal setting unit 115 may change the next intermediate goal and postpone the due date on the basis of the motivation of the subject 41 and the period taken for achieving the one previous intermediate goal. In this case, the level of the next intermediate goal after the change may be a lower level, i.e., a level easier to achieve in comparison with the next intermediate goal level before the change, for example.

Note that in this embodiment, the motivation of the subject 41 is reflected in the evaluation result of the evaluation unit 114, and the goal setting unit 115 sets the intermediate goal on the basis of the information about the evaluation result.

As another example, the goal setting unit 115 may set the intermediate goal on the basis of the information about the estimation result of the motivation estimation unit 113 together with or instead of the information about the evaluation result of the evaluation unit 114. In this case, the information about the estimation result of the motivation estimation unit 113 may be output from the motivation estimation unit 113 to the goal setting unit 115.

In addition, as another example, in the case where the information of the proficiency is required separately from the information about the motivation, the goal setting unit 115 may set the intermediate goal by using the information of the proficiency together with the information about the motivation.

The advice generation unit 116 generates a predetermined advice, and outputs the information about the generated advice to the output unit 117.

This advice may have various contents, and may be an advice including an advice content for achieving the next goal that is the ideal goal or the intermediate goal, for example.

In this embodiment, the advice generation unit 116 generates the advice on the basis of the information from the goal setting unit 115.

In this case, the advice generation unit 116 may generate the advice with reference to one or more of the output information from the exercise information acquiring unit 111, the output information from the determination unit 112, the output information from the motivation estimation unit 113, and the output information from the evaluation unit 114 together with or instead of the information from the goal setting unit 115, for example. In this case, the information referred to by the advice generation unit 116 may be output from the component that outputs that information to the advice generation unit 116, for example. This component is one or more of the exercise information acquiring unit 111, the determination unit 112, the motivation estimation unit 113, and the evaluation unit 114.

In addition, the advice generation unit 116 may generate the next advice on the basis of the historical advice, for example. This historical advice may be the one previous advice, for example.

In general, for the output advice, the advice generation unit 116 may perform one or more processes of changing the difficulty of the advice, changing the number of key points, changing the expression of the advice, and demonstrating success stories of other users. The other user may be a user who have had previous experience and success with exercises similar to those being performed by the subject 41, for example.

For example, the following considers a case where the advice generation unit 116 generates the advice for the next intermediate goal in direct or indirect consideration of at least the motivation of the subject 41.

As an example, the advice generation unit 116 may generate the advice on the basis of only the motivation.

As a specific example, the advice generation unit 116 may change and present the number of key points and the difficulty of the advice in accordance with the motivation. The difficulty of the advice may be set such that the higher the motivation, higher the difficulty, and that the lower the motivation, the lower the difficulty, for example. The number of key points may be set such that the higher the motivation, the greater the number, and that the lower the motivation, the smaller the number, for example.

Normally, the lower the difficulty of the advice, the easier to understand.

In addition, normally, the smaller the number of key points, the easier to achieve.

As another example, the advice generation unit 116 may generate the advice on the basis of the motivation and other information.

The other information may be various information, and one or more information of the intermediate goal, the current state of the subject 41, and the item for achieving the ideal goal may be used, for example. The expression of the advice may be made specific in accordance with such information.

As a specific example, an example of a marathon is described below.

When the advice generation unit 116 generates the advice in accordance with only the motivation, an expression of the advice “Run 3 km in 30 minutes two times” is used as an example, and thus the goal of the exercise can be determined.

On the other hand, when the advice generation unit 116 generates the advice in accordance with the motivation and other information, an expression of the advice “Vertical movement is 30 cm, which is too large. Keep in mind it should be 20 cm, and run 3 km in 30 minutes two times” is used as an example, and thus a more detailed item for achieving the goal is expressed. Note that the advice of this example is an advice that takes into account the current state of the subject 41, and specifically expresses what should be done further in consideration of the item to achieve the ideal goal.

In addition, when the motivation of the subject 41 is low, the advice generation unit 116 may find successful cases of a state close to the subject 41 on the basis of the current state of the subject 41 and the history of the goal achievement state of another user, and present the found cases as successful case, for example. In this manner, the motivation of the subject 41 can be increased. For successful cases of other users, this presentation may include information about a goal representing the ideal goal or the intermediate goal, the state of the other users before receiving the advice, and the period for the achievement of the goal by the other users. Other users may be subjects other than the subject 41, and may be a person other than the subject 41 who performs the same sports as the subject 41, for example.

Note that such a presentation may be performed in one or both of the case where the advice generation unit 116 generates the advice on the basis of only the motivation, and the case where the advice generation unit 116 generates the advice on the basis of the motivation and other information, for example.

With such a presentation, the advice generation unit 116 can introduce the subject 41 with low motivation to a situation where other users achieved the goal with a capability close to the capability of the subject 41 based on an advice, which can let the subject 41 to know how the other users have grown, and the like, for example.

The output unit 117 outputs the information from the advice generation unit 116 to the display device 31. In this manner, this information is displayed on the screen of the display device 31 and notified to the subject 41 and the like.

Note that in this embodiment, the output unit 117 outputs to the display device 31 the information from the advice generation unit 116, but the output unit 117 may output other information to the display device 31 to display the information on the display device 31.

In addition, this embodiment describes one display device 31 for convenience of description, but a plurality of display devices including the display device 31 may be provided in the information processing system 1, and in such a case, the output unit 117 may output the same information or different information to the two or more display devices, for example.

The storage unit 118 has a function of storing various information.

In this embodiment, the storage unit 118 starts storing the information related to the subject 41 from the initial state of the subject 41, and stores and holds the information related to the subject 41 until the subject 41 achieves the ideal goal. Such information may be information about a situation where the subject 41 has continued a predetermined sport from the initial state, achieved an intermediate goal, and finally achieved an ideal goal, for example. Such information may be referred to as history information. The history information is not limited to the information of this example, but may be any information. Note that the subject 41 may not finally achieve the ideal goal.

The history information may include one or more of the operation history input from a predetermined application, the ideal goal, the intermediate goal, the exercise information, the behavior information, the exercise-related information, the state of the subject 41, the motivation of the subject 41, the goal achievement state, the advice presented to the subject 41, and the like, for example.

The goal achievement state may be information about the number of days divided as the intermediate goal, the number of days until the achievement of the previous goal, and the like, for example.

In this embodiment, such history information is stored and held in the storage unit 118.

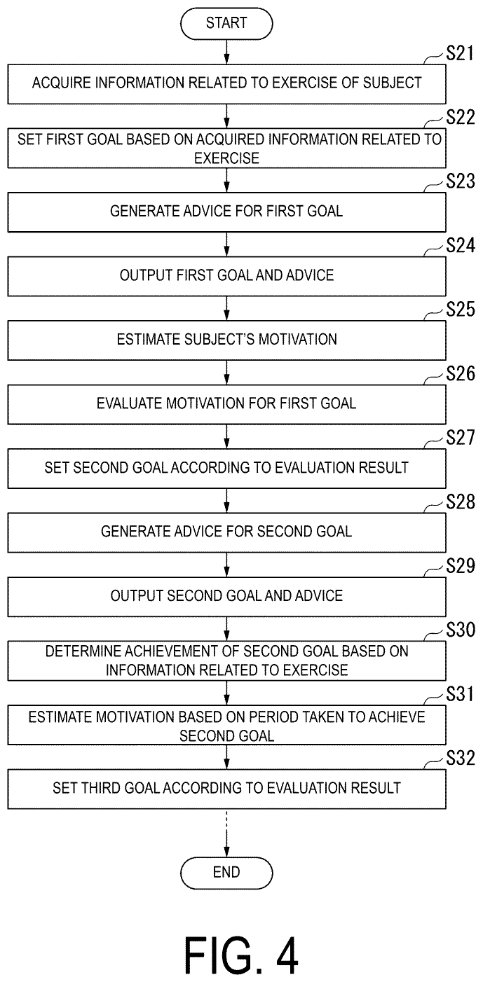

FIG. 4 illustrates an example of a procedure of a process performed in the information processing device 11 according to the embodiment.

In the example illustrated in FIG. 4, the information processing device 11 performs the processes of step S21 to step S32.

In the processing procedure of this example, the processes of step S21 to step S32 are sequentially performed.

At step S21, the exercise information acquiring unit 111 acquires the information related to the exercise of the subject 41.

At step S22, the goal setting unit 115 sets a first goal on the basis of the exercise information acquired by the exercise information acquiring unit 111. In this example, the first goal is an ideal goal.

At step S23, the advice generation unit 116 generates an advice for the first goal. Note that this advice is referred to as first advice, for example.

At step S24, the output unit 117 outputs information about the set first goal and the generated advice by the display device 31. The subject 41 can perform check and the like on the information.

At step S25, the motivation estimation unit 113 estimates the motivation of the subject 41.

At step S26, the evaluation unit 114 evaluates the motivation for the first goal.

At step S27, the goal setting unit 115 sets a second goal in accordance with the evaluation result. In this example, the second goal is the first intermediate goal.

At step S28, the advice generation unit 116 generates the advice for the second goal. Note that this advice may be referred to as second advice, for example.

At step S29, the output unit 117 outputs information about the set second goal and the generated advice by the display device 31. The subject 41 can perform check and the like on the information.

At step S30, the determination unit 112 determines the achievement of the second goal on the basis of the information related to the exercise. It is assumed here that the second goal has been achieved by the subject 41.

At step S31, the motivation estimation unit 113 estimates the motivation in accordance with the period taken for achieving the second goal.

The evaluation unit 114 evaluates the motivation for the second goal.

At step S32, the goal setting unit 115 sets a third goal on the basis of the evaluation result. In this example, the third goal is the second intermediate goal.

Note that in FIG. 4, although not illustrated in the drawing, after step S32, the information processing device 11 generates and outputs advices and as necessary sets the next goal for each goal after the third goal, and finally the first goal as the ideal goal is set so as to achieve the ideal goal.

For example, the first goal as the ideal goal may be set as a fourth goal in the third goal, or an intermediate goal may be set as one or more continuous goals including the fourth goal, and then, the first goal as the ideal goal may be finally set.

As described above, regarding the exercise of the subject 41, the information processing device 11 in the information processing system 1 according to this embodiment performs the evaluation on the motivation of the subject 41 after the advice for the predetermined goal is output, and outputs an advice different from the advice on the basis of the evaluation result, and thus the advice suitable for the motivation of the subject 41 can be provided.

For example, as in known technology, in the case where the advice is generated for the user in accordance with only the proficiency, the user may not achieve the content of the advice, and the advice of the same content may be continuously generated if the goal is not changed. As such, in known technology, if the situation where the user cannot achieve the content of the same advice continues, the motivation of the user is lowered, which may make it difficult to achieve the goal.

Conversely, the information processing device 11 according to this embodiment can present the advice obtained by changing the advice that has been presented for the goal to another advice corresponding to the motivation of the subject 41 in accordance with the evaluation result of the motivation of the subject 41.

In this manner, the information processing device 11 according to this embodiment can prevent the reduction of the motivation of the user to maintain or improve the motivation of the user.

For example, the information processing device 11 according to this embodiment can estimate the motivation of the subject 41 on the basis of the information about the exercise and the behavior of the subject 41 subject to instruction, and provide a notification of the advice by setting an intermediate goal with the difficulty that is easy to achieve for the subject 41 in accordance with the estimated motivation.

In this manner, the information processing device 11 according to this embodiment can support the subject 41 to easily perform the exercise while maintaining the motivation for achieving each goal, and encourage the subject 41 to actively perform the exercise and challenge to achieve the goal.

As a configuration example, the information processing device 11 includes the goal setting unit 115, the advice generation unit 116, the output unit 117, the motivation estimation unit 113, and the evaluation unit 114.

The goal setting unit 115 sets the first goal for the exercise of the subject 41. The first goal is an ideal goal.

The advice generation unit 116 generates a first advice for the first goal.

The output unit 117 outputs the first goal and the first advice.

The motivation estimation unit 113 estimates the motivation of the subject 41.

The evaluation unit 114 evaluates the motivation of the subject 41 for the first goal.

The advice generation unit 116 generates a second advice different from the first advice on the basis of the evaluation result of the evaluation unit 114.

The output unit 117 outputs the second advice.

Thus, the information processing device 11 can present the second advice based on the evaluation result of the motivation of the subject 41 for the first goal to the subject 41, and can provide the advice suitable for the motivation of the subject 41.

As a configuration example, the information processing device 11 includes the exercise information acquiring unit 111.

The exercise information acquiring unit 111 acquires the information related to the exercise of the subject 41 from the sensor attached to the subject 41. In this embodiment, this sensor is the sensor of the sensor unit 21.

The goal setting unit 115 sets the first goal on the basis of the information related to the exercise of the subject 41. Thus, the information processing device 11 can set a goal suitable for the capability of the exercise of the subject 41 and the like as the first goal.

As a configuration example, in the information processing device 11, the motivation estimation unit 113 estimates the motivation of the subject 41 based on the information related to the exercise of the subject 41.

Thus, the information processing device 11 can estimate the motivation reflecting the actual exercise situation of the subject 41.

As a configuration example, the motivation estimation unit 113 estimates the motivation of the subject 41 based on a time when the subject 41 performed the exercise, the number of times the subject 41 performed the exercise, and an exercise intensity of the exercise performed by the subject 41 that are included in the information related to the exercise of the subject 41. Thus, the information processing device 11 can estimate the motivation reflecting the exercise time, the number of times of the exercise, the exercise intensity of the subject 41.

Note that the exercise frequency may be used together with or instead of the number of times of the exercise.

As a configuration example, in the information processing device 11, the motivation estimation unit 113 estimates the motivation of the subject 41 based on the number of times the subject 41 viewed the first advice and the first goal output to an external device.

Thus, the information processing device 11 can estimate the motivation of the subject 41 based on the behavior of the subject 41 viewing the advice and the like. Normally, it is estimated that the greater the number of times of the viewing, the higher the motivation.

Note that in this embodiment, the display device 31 is an example of the external device.

As a configuration example, the goal setting unit 115 sets a second goal different from the first goal based on the evaluation result. The second goal is an intermediate goal, and is the first intermediate goal in this case.

The second advice is an advice for the second goal.

Thus, the information processing device 11 can prevent subject 41 from giving up on exercise in the process by setting an intermediate goal with a middle level before the achievement of the ideal goal.

As a configuration example, the determination unit 112 is provided.

The determination unit 112 is configured to determine a difference between a current state of the subject 41 and a state set by the first goal.

The goal setting unit 115 sets the second goal based on a result of an evaluation performed by the evaluation unit 114 based on a determination result of the determination unit and the motivation of the subject 41.

Thus, the information processing device 11 can set an intermediate goal reflecting the degree of the motivation of the subject 41 and the difference between the current state of the subject 41 and the ideal goal state, for example.

As a configuration example, in the information processing device 11, the goal setting unit 115 sets a level of the second goal that is set when the motivation estimation unit 113 estimates that the motivation of the subject 41 is high to a level closer to a level of the first goal than a level of the second goal that is set when the motivation estimation unit 113 estimates that the motivation of the subject 41 is low.

Thus, when the motivation of the subject 41 is high, the information processing device 11 can set an intermediate goal with a level closer to the ideal goal than when the motivation is low. That is, it is estimated that the higher the motivation of the subject 41, the higher the intermediate goal to be achieved.

Note that the degree of the motivation of the subject 41 may not be determined in a continuous value, but may be determined in discrete values such as high, middle and low, for example.

As a configuration example, the determination unit 112 determines whether a current state of the subject 41 reached a state of the second goal based on the information related to the exercise of the subject 41.

The motivation estimation unit 113 estimates the motivation of the subject 41 based on a period taken for the subject 41 to achieve the second goal.

The evaluation unit 114 evaluates the motivation of the subject 41 for the second goal.

The goal setting unit 115 sets a third goal different from the second goal based on a result of the motivation evaluated by the evaluation unit 114. The third goal is an intermediate goal, and is the second intermediate goal in this case.

In this manner, the information processing device 11 can set the second intermediate goal reflecting the motivation of the subject 41 for the first intermediate goal, and thus can sequentially set the intermediate goal suitable for the motivation of the subject 41.

While the goal setting unit 115 sets one or more intermediate goals in this embodiment, the intermediate goal may not be set, and the acquisition of the exercise information, the various determinations, the estimation of the motivation, the evaluation, the generation and output of advices and the like may be performed with only the ideal goal set in the information processing device 11, for example.

In addition, in the information processing device 11 according to this embodiment, it is possible to use a configuration of performing processes using machine learning or a configuration of performing processes using parameters such as a predetermined computation expression and threshold value without using machine learning in the exercise information acquiring unit 111, the determination unit 112, the motivation estimation unit 113, the evaluation unit 114, the goal setting unit 115, the advice generation unit 116, and the like, for example.

Note that in this embodiment, the movement of the subject 41 for performing sports is described using a term “exercise”, but the exercise performed by the subject 41 to achieve an ideal goal of a specific type of exercise may be referred to as “practice” or the like, for example. The specific type of exercise may be an exercise of specific sports, or an exercise of moving a specific body part in a specific manner, for example.

In this case, the exercise information, the exercise-related information, the exercise time, the number of times of the exercise, the exercise frequency, the exercise intensity, and the exercise load in this embodiment may be referred to as practice information, practice-related information, practice time, practice the number of times, practice frequency, practice intensity, and practice load, respectively.

A program for realizing the function of a given component in a given device described above may be recorded on a computer-readable storage medium, and the program may be read and executed by a computer system. The “computer system” as used herein is assumed to include hardware such as an operating system or peripheral devices. The “computer-readable storage medium” is a storage device such as a portable medium such as a flexible disk, a magneto-optical disk, a read only memory (ROM), a compact disc (CD)-ROM, and a hard disk built into a computer system. The “computer-readable storage medium” is assumed to include a medium that holds a program for a certain period of time, such as a volatile memory provided inside of a computer system serving as a server or a client when the program is transmitted via a network such as the Internet or a communication line such as a telephone line. The volatile memory may be a RAM. The storage medium may be a non-transitory storage medium.

The program described above may be transmitted from a computer system storing this program in a storage device or the like via a transmission medium or using transmission waves in a transmission medium to another computer system. The “transmission medium” from which the program is transmitted refers to a medium having a function of transmitting information, such as a network such as the Internet or a communication line such as a telephone line.

The program described above may be a program for realizing a part of the above-described functions. The program described above may be a so-called difference file, which can realize the above-described functions in combination with a program already recorded in the computer system. The difference file may be called a difference program.

The function of a given component in a given device described above may be realized by a processor. Each of the processes in the embodiment may be realized by a processor that operates on the basis of information such as a program and a computer-readable storage medium that stores information such as a program. In the processor, the function of each unit may be realized by individual hardware, or the function of each unit may be realized by integrated hardware. The processor may include hardware, and the hardware may include at least one of a circuit for processing digital signals and a circuit for processing analog signals. The processor may be configured using one of or both of one or more circuit devices or one or more circuit elements mounted on a circuit board. For the circuit device, an integrated circuit (IC) or the like may be used, and for the circuit element, a resistor, a capacitor, or the like may be used.

The processor may be a CPU. However, the processor is not limited to the CPU, and it may be possible to use various processors such as a graphics processing unit (GPU) or a digital signal processor (DSP). The processor may be a hardware circuit using an application-specific integrated circuit (ASIC). The processor may be configured by a plurality of CPUs or may be configured by a hardware circuit including a plurality of ASICs. The processor may be configured by a combination of a plurality of CPUs and a hardware circuit including a plurality of ASICs. The processor may include one or more of an amplifier circuit, a filter circuit, and the like for processing analog signals.

These are detailed descriptions of the embodiment with reference to the drawings. However, the specific configurations are not limited to those of this embodiment, and include designs and the like without departing from the gist of the present disclosure.

### Supplementary Notes

The following describes Configuration Example 1 to Configuration Example 10.

Note that a lower configuration example may or may not be applied to a higher configuration example.

In addition, a lower configuration example applicable to any one of two or more higher configuration examples may be applied to any configuration example of the two or more higher configuration examples. In other words, two or more application examples may be generated, and configuration examples lower than the lower configuration examples described above may be applied to any application example of the two or more application examples.

### Configuration Example 1

An information processing device, including:
a goal setting unit configured to set a first goal for an exercise of a subject;
an advice generation unit configured to generate a first advice for the first goal;
an output unit configured to output the first goal and the first advice;
a motivation estimation unit configured to estimate a motivation of the subject; and
an evaluation unit configured to evaluate the motivation of the subject for the first goal, in which
the advice generation unit generates a second advice different from the first advice based on a result of the evaluation by the evaluation unit, and
the output unit outputs the second advice.

### Configuration Example 2

The information processing device according to Configuration Example 1, including an exercise information acquiring unit configured to acquire information related to the exercise of the subject from a sensor attached to the subject, in which
the goal setting unit sets the first goal based on the information related to the exercise of the subject.

### Configuration Example 3

The information processing device according to Configuration Example 1, including an exercise information acquiring unit configured to acquire the information related to the exercise of the subject from a sensor attached to the subject, in which
the motivation estimation unit estimates the motivation of the subject based on the information related to the exercise of the subject.

### Configuration Example 3A

Configuration Example 2 and Configuration Example 3 may be combined.

In this case, the exercise information acquiring unit of Configuration Example 2 and the exercise information acquiring unit of Configuration Example 3 may be common components.

### Configuration Example 4

The information processing device according to Configuration Example 3 or 3A, in which the motivation estimation unit estimates the motivation of the subject based on a time when the subject performs the exercise, the number of times the subject performs the exercise, and an exercise intensity of the exercise performed by the subject, included in the information related to the exercise of the subject.

### Configuration Example 5

The information processing device according to any one of Configuration Examples 1 to 4 and 3A, in which the motivation estimation unit estimates the motivation of the subject based on the first goal output to an external device and the number of times the subject views the first advice.

### Configuration Example 6

The information processing device according to any one of Configuration Examples 1 to 5 and 3A, in which the goal setting unit sets a second goal different from the first goal based on a result of the evaluation, and the second advice is an advice for the second goal.

### Configuration Example 7

The information processing device according to Configuration Example 6, including a determination unit configured to determine a difference between a current state of the subject and a state set by the first goal, in which
the goal setting unit sets the second goal based on a result of the evaluation performed by the evaluation unit based on the motivation of the subject and a result of the determination by the determination unit.

### Configuration Example 8

The information processing device according to Configuration Example 6 or 7, in which the goal setting unit sets a level of the second goal set when the motivation estimation unit estimates that the motivation of the subject is high to a level closer to a level of the first goal than a level of the second goal set when the motivation estimation unit estimates that the motivation of the subject is low.

### Configuration Example 9

The information processing device according to Configuration Example 7, in which the determination unit determines whether the current state of the subject reaches a state of the second goal, based on information related to an exercise of the subject,
the motivation estimation unit estimates the motivation of the subject based on a period required for the subject to achieve the second goal,
the evaluation unit evaluates the motivation of the subject for the second goal, and
   the goal setting unit sets a third goal different from the second goal based on a result of the evaluation of the motivation by the evaluation unit.

### Configuration Example 9A

Configuration Example 9 and Configuration Example 8 may be combined.

It is also possible to provide a program storage medium that stores a program executed by a processor in the information processing device as described above.

### Configuration Example 10

A program storage medium configured to store a program, the program causing a computer to execute:
a first goal setting function of setting a first goal for an exercise of a subject;
a first advice generation function of generating a first advice for the first goal;
a first output function of outputting the first goal and the first advice;
a first estimation function of estimating a motivation of the subject;
a first evaluation function of evaluating the motivation of the subject for the first goal;
a second advice generation function of generating a second advice different from the first advice based on a result of the evaluation by the first evaluation function; and
a second output function of outputting the second advice.

What is claimed is:

1. An information processing device, comprising:
a goal setting unit configured to set a first goal for an exercise of a subject;
an advice generation unit configured to generate a first advice for the first goal;
an output unit configured to output the first goal and the first advice;
a motivation estimation unit configured to estimate a motivation of the subject; and
an evaluation unit configured to evaluate the motivation of the subject for the first goal, wherein
the advice generation unit generates a second advice different from the first advice based on a result of the evaluation by the evaluation unit, and
the output unit outputs the second advice.

2. The information processing device according to claim 1, further comprising an exercise information acquiring unit configured to acquire information related to the exercise of the subject from a sensor attached to the subject, wherein
the goal setting unit sets the first goal based on the information related to the exercise of the subject.

3. The information processing device according to claim 1, further comprising an exercise information acquiring unit configured to acquire the information related to the exercise of the subject from a sensor attached to the subject, wherein
the motivation estimation unit estimates the motivation of the subject based on the information related to the exercise of the subject.

4. The information processing device according to claim 3, wherein the motivation estimation unit estimates the motivation of the subject based on a time when the subject performs the exercise, the number of times the subject performs the exercise, and an exercise intensity of the exercise performed by the subject, included in the information related to the exercise of the subject.

5. The information processing device according to claim 4, wherein the motivation estimation unit estimates the motivation of the subject based on the first goal output to an external device and the number of times the subject views the first advice.

6. The information processing device according to claim 1, wherein
the goal setting unit sets a second goal different from the first goal based on a result of the evaluation, and
the second advice is an advice for the second goal.

7. The information processing device according to claim 6, further comprising a determination unit configured to determine a difference between a current state of the subject and a state set by the first goal, wherein
the goal setting unit sets the second goal based on a result of the evaluation performed by the evaluation unit based on the motivation of the subject and a result of the determination by the determination unit.

8. The information processing device according to claim 7, wherein the goal setting unit sets a level of the second goal set when the motivation estimation unit estimates that the motivation of the subject is high to a level closer to a level of the first goal than a level of the second goal set when the motivation estimation unit estimates that the motivation of the subject is low.

9. The information processing device according to claim 7, wherein
the determination unit determines whether the current state of the subject reaches a state of the second goal, based on information related to an exercise of the subject,
the motivation estimation unit estimates the motivation of the subject based on a period required for the subject to achieve the second goal,
the evaluation unit evaluates the motivation of the subject for the second goal, and
the goal setting unit sets a third goal different from the second goal based on a result of the evaluation of the motivation by the evaluation unit.

10. A program storage medium configured to store a program, the program causing a computer to execute:
a first goal setting function of setting a first goal for an exercise of a subject;
a first advice generation function of generating a first advice for the first goal;
a first output function of outputting the first goal and the first advice;
a first estimation function of estimating a motivation of the subject;
a first evaluation function of evaluating the motivation of the subject for the first goal;
a second advice generation function of generating a second advice different from the first advice based on a result of the evaluation by the first evaluation function; and
a second output function of outputting the second advice.
